(19) United States
(12) Patent Application Publication
(10) Pub. No.: US 2025/0359800 A1
(43) Pub. Date: Nov. 27, 2025

(54) ELECTROMYOGRAPHY DEVICES AND METHODS EMPLOYING PROCESSING OF MOTOR UNIT ACTION POTENTIALS VIA NEUROMORPHIC COMPUTING TO DECODE INTENTION OR PREDICT MOTOR FUNCTION

(71) Applicant: Battelle Memorial Institute, Columbus, OH (US)

(72) Inventors: Nicholas J. Tacca, Columbus, OH (US); Eric Meyers, Columbus, OH (US); Ian W. Baumgart, Albany, OH (US)

(21) Appl. No.: 19/215,470

(22) Filed: May 22, 2025

Related U.S. Application Data
(60) Provisional application No. 63/650,469, filed on May 22, 2024.

(51) Int. Cl. A61B 5/296; A61B 5/00; A61B 5/256; A61B 5/30; A61B 5/389

(52) U.S. Cl. CPC A61B 5/296; A61B 5/256; A61B 5/30; A61B 5/389; A61B 5/7228

(57) ABSTRACT

A garment is worn on an anatomical region, with electrodes arranged on the garment to contact skin of the anatomical region. An EMG amplifier measures analog EMG data, and analog circuitry decomposes the analog EMG data into MUAPs. The analog circuitry may include an analog matrix processor performing analog matrix multiplication to transform the analog EMG data into source signals, an analog squarer circuit computing power signals by squaring the source signals, and delta sigma analog-to-digital converters converting the power signals to analog spike signals. The analog circuitry may include a neuromorphic chip to transform the analog EMG data into analog spike signals using blind source separation. A neuromorphic chip may process the analog spike signals to determine volitional intent using spiking neural networks (SNN), and/or perform a neuromuscular debilitation assessment based on the analog spike signals or encoded spike train

This application claims the benefit of U.S. provisional application Ser. No. 63/650,469 filed May 22, 2024, which is incorporated herein by reference in its entirety.

### BACKGROUND

The following relates to the electromyography arts, neuromuscular electrical stimulation arts, neuromuscular therapy arts, neuromuscular rehabilitation arts, virtual reality arts, augmented reality arts, and to the like.

The following relates to improvements in electromyography (EMG) measurement and analysis, and to applications of same in diverse fields such as: neuromuscular electrical stimulation (NMES) guided by EMG signals; EMG-based assessment of neuromuscular debilitation due to spinal cord injury (SCI), stroke, traumatic brain injury (TBI), or other pathologies such as Parkinson's disease; neuromuscular therapy and/or rehabilitation performed using or guided by EMG measurements; EMG-guided muscle tremors suppression; virtual reality (VR) or augmented reality (AR) systems utilizing EMG measurements to monitor participant activity and/or guide VR or AR content presentation; sports performance or technique analysis; injury recovery; fatigue assessment; and like applications.

In performing such tasks, accurate measurement of EMG signals can be challenging. For example, artifacts can be introduced into the EMG signals by diverse sources such as electrode impedance changes caused by shifting of the electrodes or sweat, radio frequency interference (RFI) from local electronics, and so forth. If EMG measurements are interleaved with NMES pulses, for example in EMG-guided NMES applications, then the NMES pulses can also interfere with the EMG signal measurement.

Various improvements are disclosed herein.

### BRIEF SUMMARY

In accordance with some illustrative embodiments disclosed herein, an electromyography (EMG) measurement device includes a garment configured to be worn on an anatomical region of an associated wearer, a plurality of electrodes arranged on the garment to contact skin of the anatomical region when the garment is worn on the anatomical region of the associated wearer, and electronics configured to perform motor unit action potential (MUAP) decomposition on the EMG data. The electronics include an EMG amplifier operatively connected with the electrodes to measure analog EMG data emanating from the anatomical region, and circuitry configured to decompose the analog EMG data into MUAPs. In some embodiments, the circuitry includes an analog matrix processor configured to perform analog matrix multiplication to transform the analog EMG data into source signals, an analog squarer circuit configured to compute power signals by squaring the source signals output by the analog matrix processor, and delta sigma analog-to-digital converters configured to convert the power signals to analog spike signals. In some embodiments, the circuitry includes a neuromorphic chip configured to transform the analog EMG data into analog spike signals using blind source separation. In some embodiments, a neuromorphic chip is configured to process the analog spike signals to determine volitional intent using a spiking neural network (SNN), and/or to perform a neuromuscular debilitation assessment based on the analog spike signals.

In accordance with some illustrative embodiments disclosed herein, a motor cortical activity estimation method includes measuring EMG data emanating from an anatomical region, decomposing the EMG data into MUAPs, and determining motor unit (MU) synergies representing motor cortical activity from the MUAPs using a spiking neural network (SNN) encoder.

In accordance with some illustrative embodiments disclosed herein, an EMG data processing device includes analog source separation circuitry configured to transform analog EMG data into source signals using blind source separation, and analog spike signal generation circuitry configured to convert the source signals into analog spike signals. At least some of the analog spike signals correspond to motor unit action potentials (MUAPs). In some embodiments, the analog source separation circuitry includes an analog matrix processor configured to perform analog matrix multiplication to transform the analog EMG data into source signals, and an analog squarer circuit configured to compute power signals by squaring the source signals output by the analog matrix processor, and the analog spike signal generation circuitry includes delta sigma analog-to-digital converters configured to convert the power signals to analog spike signals. In some embodiments, the analog source separation circuitry and the analog spike signal generation circuitry include a neuromorphic chip configured to transform the analog EMG data into analog spike signals corresponding to source signals using blind source separation. In some embodiments, the EMG data processing device further includes analog filtering circuitry configured to select the analog spike signals corresponding to MUAPs from the analog spike signals output by the analog spike signal generation circuitry, for example based on pulse-to-noise ratios (PNRs) of the analog spike signals output by the analog spike signal generation circuitry.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows an electromyography (EMG) measurement system, together with an optional neuromuscular electrical stimulation (NMES) capability.

FIG. 2 diagrammatically shows a flowchart of a nonlimiting illustrative embodiment of motor unit action potential (MUAP) decomposition using blind source separation (BSS).

FIG. 3 diagrammatically shows a flowchart of a nonlimiting illustrative embodiment of for performing EMG filtering using BSS.

FIG. 4 diagrammatically shows a flowchart of a nonlimiting illustrative embodiment for performing MUAP decomposition by BSS.

FIGS. 5 and 6 diagrammatically shows further nonlimiting illustrative embodiments of an EMG measurement system with optional NMES capability.

FIG. 7 diagrammatically shows the analog matrix processor of the system of FIG. 5 in further detail.

FIG. 8 diagrammatically shows a flowchart for inferring motor cortical activity based on the resulting MU spiking input to muscles.

### DETAILED DESCRIPTION

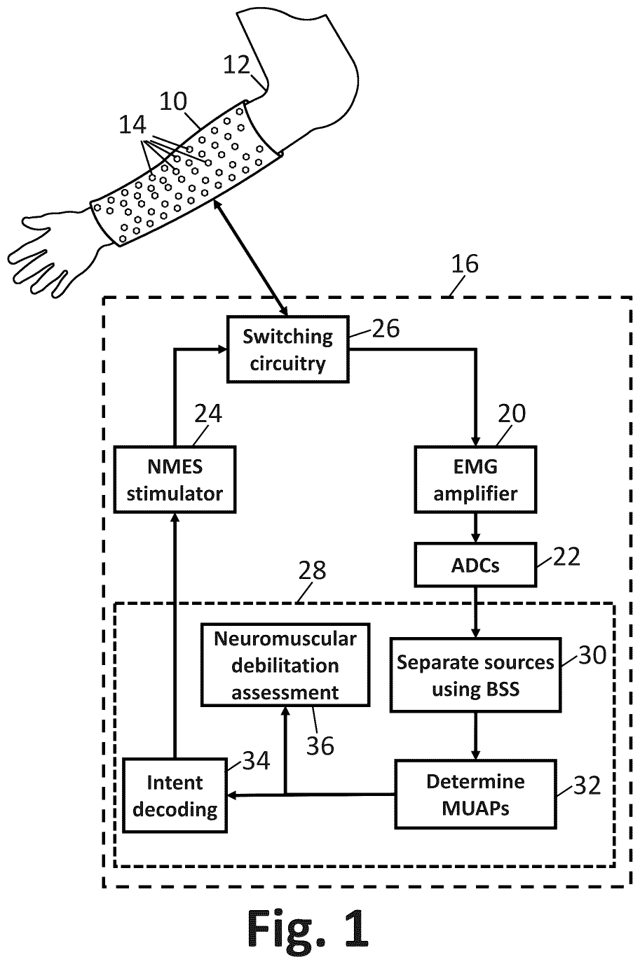

With reference to FIG. 1, an electromyography (EMG) measurement system is shown, which in the illustrative example also includes an optional neuromuscular electrical stimulation (NMES) capability. The EMG measurement system includes a garment 10 that is wearable on an anatomical region 12, and that includes a plurality of electrodes 14 arranged to contact skin of the anatomical region 12 when the garment is worn on the anatomical region. The illustrative garment 10 is a sleeve 10 worn on an arm 12. The garment 10 may be made of a cloth, textile, leather, polyester, or other material, and is sized and shaped to be worn on the anatomical region 12 from which EMG is to be measured. The garment 10 may more generally, for example, be a sleeve that is sized and shaped to be worn on an arm, a leg, a wrist, an ankle, an arm and a wrist, a leg and an ankle, a torso, or so forth. By way of some further examples, suitable garments for a hand would include, for example, a glove or mitten. Suitable garments for a foot would include, for example, a sock or boot. The glove, mitten, sock, or boot can be extended over the wrist or ankle to provide a garment for a wrist and hand or for an ankle and foot, or further extended to provide a garment for an arm and wrist and hand or for a leg and ankle and foot. These are merely non-limiting illustrative examples.

The sizing of the garment 10 is suitably subject-specific to account for different anatomies of different persons. Alternatively, the garment 10 may be designed to be adjustable for anatomical differences between persons—for example, the illustrative sleeve 10 could employ a wrap-around arrangement with Velcro that can be adjustably wrapped around arms of different diameters. The plurality of electrodes 14 are disposed on the inside of the garment 10 to contact the skin of the anatomical region 12 when the garment 10 is worn on the anatomical region 12. Note that FIG. 1 illustrates the garment 10 as transparent to reveal the underlying electrodes 14, but more typically the garment will be translucent or opaque. The electrodes 14 are connected by wires (possibly woven into the garment 10), circuitry of flexible printed circuit boards, and/or so forth to connect with associated electronics 16. The various components of the electronics 16 may be integrated with the garment 10, or separate from the garment 10 and connected with the electrodes 14 by suitable electrical wires or cables or the like. Typically, the electrodes 14 are surface electrodes, i.e., transcutaneous electrodes; however, embodying the electrodes 14 as needle electrodes or the like is also contemplated. In some embodiments, the garment 10 is an elastic garment whose elasticity provides compressive force holding the electrodes 14 firmly against the skin of the wearer. Such garment elasticity can also in some specific implementations facilitate the garment 10 being wearable on arms (or other target anatomical region 12) of different sizes. The electrodes 14 are designed to provide good electrical contact with the skin of the anatomical region 12. For example, the electrodes 14 may be electrogel discs, or may comprise an electrically conductive polymer electrode material such as a mixed ionic-electronic conducting (MIEC) material, or so forth. Optionally, the garment 10 may further include other devices such as one or more inertial measurement unit (IMU) devices (not shown) such as an accelerometer, gyroscope, or the like, to provide information on the spatial orientation of the sleeve 10 (and hence of the anatomical region 12).

The electrodes 14 are used to measure EMG signals produced by the anatomical region 12. The EMG signal measurements are potential difference measurements between pairs of electrodes 14, where the pairs of electrodes are pairs of electrodes of the array or, in a monopolar configuration, each pair is an electrode of the array and a common reference electrode. Each such pair of electrodes is referred to herein as an EMG channel. To this end, the electronics 16 include an EMG amplifier 20, which may for example comprise an operational amplifier (op-amp) based amplifier circuit. It will be appreciated that the EMG amplifier 20 is a multi-channel amplifier, e.g. each EMG channel (corresponding to an electrode 14: reference-electrode pair, or to a pair of electrodes 14) is separately received and amplified in parallel by the multi-channel EMG amplifier 20. Preferably, the outputs of the multichannel EMG amplifier 20 are digitized by analog-to-digital converters (ADCs) 22. By way of nonlimiting illustrative example, the combination of the multichannel EMG amplifier 20 and multichannel ADC 22 can be embodied as an Intan EMG amplifier (available from Intan Technologies, Los Angeles, California, USA).

The measured EMG can be utilized in various ways. For example, if the subject is suffering neuromuscular debilitation due to spinal cord injury (SCI), stroke, traumatic brain injury (TBI), or pathologies such as Parkinson's disease, then the EMG can be used to assess the extent to which the motor cortex of the subject's brain is able to transmit motor control neural signals to muscles of the anatomical region 12, and the accuracy of such neural signal transmission if present (e.g., if the subject's volition is to move the index finger then do the transmitted neural signals reach the muscles that cause movement the of the index finger, or are the neural signals mis-transmitted to different muscles due to the neuromuscular debilitation). As another example, if the EMG measurement system is deployed in a virtual reality (VR) or augmented reality (AR) system, then the measured EMG can be used to monitor participant activity, guide the VR or AR content presentation, or so forth. These are some nonlimiting illustrative examples of uses the measured EMG in various applications.

With continuing reference to FIG. 1, the EMG measurement system optionally further includes NMES capability, that is, the ability to apply neuromuscular electrical stimulation to the anatomical region 12 using the electrodes 14. To this end, the electronics 16 further include an NMES stimulator 24. NMES may be applied for various reasons, such as (but not limited to): providing functional electrical stimulation (FES); suppressing muscular tremors; promoting regeneration of damaged nerves; inducing somatosensation (e.g., the sensation of touch, raindrops, an arachnid crawling across the skin, or so forth); and/or et cetera. The configuration of the applied NMES (e.g., which subset of the electrodes 14 apply the NMES, the magnitude of the applied NMES, which may vary spatially over the skin of the anatomical region 12, and so forth) may optionally be guided by the measured EMG, after suitable analysis of the EMG. For example, an SCI patient may have residual motor neuron connectivity between the motor cortex and the musculature of the target anatomy 12, but this motor neuron connectivity may be insufficient to cause the muscle contraction necessary for volitional control of the anatomical region 12. In such a case, the residual motor neuron connectivity may be detected as measured EMG at the muscles intended to be contracted, and FES can then be applied to cause the muscles to actually contract thereby moving the anatomical region 12 in accordance with the volitional intent of the wearer of the garment 10.

To implement the optional NMES capability, the electronics 16 further includes an NMES stimulator 24. To enable switching between applying NMES using the NMES stimulator 24 and receiving EMG measurements via the EMG amplifier 20, suitable switching circuitry 26 is provided, including solid state relays, high voltage field effect transistor (FET) components, and so forth, to enable the same set of electrodes 14 to switch between applying NMES stimulation and measuring EMG. (It is noted that if the EMG measurement system does not include NMES capability, then both the NMES stimulator 24 and the switching circuitry 26 may be omitted.) to perform NMES, the NMES stimulator 24 generates suitable electrical pulses that are applied to the anatomical region 12 (or a selected portion thereof) by a selected subset of the electrodes 14. In some nonlimiting illustrative embodiments, the applied NMES may comprise NMES pulse waveforms including monophasic and/or biphasic pulses with a voltage between 80 to 300 Volts inclusive or higher. In one specific example, the NMES pulse waveform is a monophasic pulse with a peak current of 0-20 mA which is modulated to vary strength of muscle contraction, frequency of 50 Hz, and a pulse width duration of 500 ms. Again, these are non-limiting illustrative examples. Analogously to the EMG amplifier 20, it will be appreciated that the NMES stimulator 24 is a multichannel NMES stimulator that can in general independently apply different NMES to different channels (where a channel corresponds to an electrode 14: reference-electrode pair, or to a pair of electrodes 14).

Extraction of useful information from the digitized EMG data output by the amplifier 20 and ADCs 22 is challenging. The EMG signal is typically weak, and numerous artifact sources may be present, such as electrode impedance changes caused by shifting of the electrodes 14 or sweat, radio frequency interference (RFI) from local electronics, and so forth. If the EMG measurements are interleaved with NMES pulses applied by the NMES stimulator 24, for example in EMG-guided NMES applications, then the NMES pulses can also interfere with the EMG signal measurement.

To isolate and suppress or remove artifacts from the EMG data, the electronics 16 may further include a computer, microprocessor, or other electronic processor 28 that is programmed to perform blind source separation (BSS) processing 30 of the EMG signal to separate sources in a source space. The separated sources are expected to include motor units (i.e., MUAPs), but may also include some artifact sources. In some nonlimiting illustrative embodiments, the BSS is done using filters computed using approximate joint diagonalization of covariance (AJDC) matrices (i.e., AJDC filters). The illustrative AJDC filters used herein are an example of a second order blind source separation (BSS) method. While AJDC filters are described in the nonlimiting illustrative examples for the source separation 30 of the EMG data using BSS, other types of BSS methods can be used for the operation 30, such as independent component analysis (ICA), principal component analysis (PCA) methods, non-negative matrix factorization methods, low-complexity coding and decoding methods, or so forth are contemplated for performing the EMG filtering.

The separated sources resulting from the BSS 30 may be used for various purposes. In FIG. 1, the electronic processor 28 is further programmed to perform processing 32 of the separated sources into motor unit action potentials (MUAPs). The BSS 30 and further MUAP decomposition processing 32 can be done in real-time to provide MUAP activity in real time.

In the illustrative example of FIG. 1, two nonlimiting examples of applications utilizing the MUAP activity are shown. In one example, the electronic processor 28 is further programmed to perform intent decoding processing 34 to determine volitional intent of the user (i.e., the person wearing the garment 10). In this example application, the user may be an SCI patient who has residual motor neuron connectivity between the user's motor cortex and the user's musculature of the target anatomy 12, but this residual motor neuron connectivity is insufficient to cause the muscle contraction necessary for volitional control of the anatomical region 12. The intent decoding 34 determines the user's volitional intent by detecting which motor units are receiving the residual motor neuron signals (as manifested by MUAP activity of those muscle units and/or associated motor neurons obtained by the processing 30 and 32), and the NMES stimulator 24 then applies functional electrical stimulation to the identified muscles via the electrodes 14 to cause the anatomical region 12 to perform the movement volitionally intended by the user. In this application, it will be appreciated that the artifact removal processing 30 advantageously removes artifacts produced by the NMES applied during stimulation time intervals that are interleaved with EMG measurement intervals during which the EMG is measured and processed (including the filtering 30 and MUAP activity extraction 32 and intent decoding 34).

In another illustrative application utilizing the MUAP activity, the electronic processor 28 is further programmed to perform neuromuscular debilitation assessment 36. This can take various forms. For example, in one approach the user (who is suffering from neuromuscular debilitation due to SCI, stroke, TBI, or another pathology such as Parkinson's disease) is asked to perform a movement of the anatomical region 12. The user makes the effort but is unable to perform the movement, or performs the movement poorly. The MUAP activity obtained by the processing 30 and 32 during this effort is processed by the assessment processing 36 to determine relevant information such as firing characteristics, the strength of motor neural signals delivered to the anatomical region 12 during the user's effort, information on how motor units fire with respect to one another (MU firing coherence), as well as information on how accurately those motor neural signals are targeted to the correct (versus incorrect) muscles.

The applications 34 and 36 diagrammatically shown in FIG. 1 are nonlimiting examples. More generally, the filtered EMG data produced by the filtering 30 and the MUAP activity produced by the optional MUAP decomposition 32 can be used in various applications such as: EMG-guided NMES (i.e., using volitional intent obtained from the measured EMG via intent decoding 34); EMG-based assessment 36 of neuromuscular debilitation due to SCI, stroke, TBI, Parkinson's disease, or so forth; neuromuscular therapy and/or rehabilitation performed using or guided by EMG measurements; EMG-guided muscle tremors suppression; VR or AR systems utilizing EMG measurements to monitor participant activity and/or guide VR or AR content presentation; and like applications.

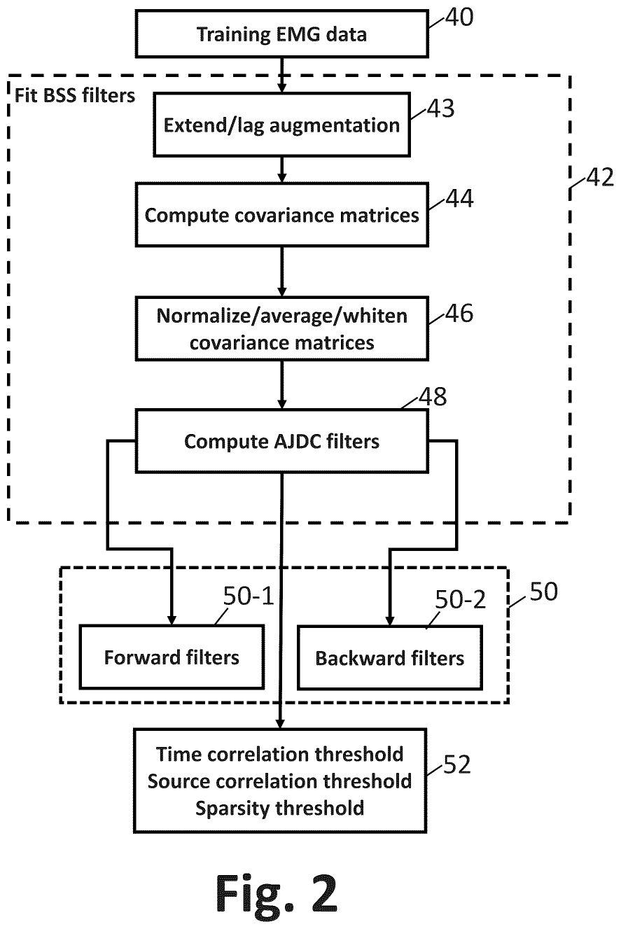

With reference now to FIG. 2, the BSS filters used in the EMG filtering 30 are fitted using a method shown in FIG. 2.

Sampled data from training EMG data 40 (or the full training EMG dataset 40) containing both clean training EMG data and training EMG data with artifacts is used for fitting the filter. The training EMG data 40 may be within subject (i.e., all the training EMG data 40 may be measured for a single subject, either in a single session or collected over multiple sessions) to fit AJDC filters tailored for that subject; or, the training EMG data 40 may be acquired for multiple subjects, and/or over multiple sessions, to fit AJDC filters that are generalized for the cohort of subjects represented by the training EMG data 40.

In an operation 42, the BSS filters to be used in the BSS 30 of FIG. 1 are fitted. By way of illustration, FIG. 2 illustrates a nonlimiting illustrative implementation of the BSS filter fitting 42 in which AJDC matrices are used in the BSS method. In an optional operation 43, an extend/lag procedure is initially applied to the EMG data 40 to enhance separability of sources and subsequently improve decoding performance. In addition to extending, the operation 43 introduces a lag by which the mixture is extended. For example, given an EMG signal (n_samp×n_chan×bin_len), the data augmentation is applied such that the final result yields a signal (n_samp×n_chan*ext_factor×bin_len). Each extension is lagged based on a desired input delay. By setting the delay/lag to be greater than 1, this advantageously enhances time information obtainable with a reduced number of extensions, thereby advantageously decreasing computation time while retaining more time-delayed extensions. In the illustrative examples, the optional extend/lag procedure 43 is applied prior to feature calculation (or, in some alternative embodiments, after feature calculation) to augment the EMG data 40 for input to decoders in real-time. Prior to the augmentation 43, it may be advantageous to concatenate one or more previous bins with the current bin to provide additional time information for the extend and lag augmentation procedure 43. This can provide a performance boost to the decoding models. Additionally, the extend/lag 43 may be used prior to blind source separation techniques to find additional sources that might otherwise be missed with smaller extension factors. For the EMG data 40, additional motor units can be decomposed by applying the extend/lag data augmentation 43 prior to doing blind source separation of the signal.

The optional extend/lag procedure 43 allows for higher resolution blind source separation, while retaining speed and efficiency, and can be readily incorporated into existing decoding pipelines. By including delayed extensions as input to decoders, the models can incorporate time information. Additionally, when augmenting data prior to feature calculation, the features can incorporate the time information directly, which can boost decoding performance.

Using extensions with single delays can be advantageous for blind source separation and decoding. However, including additional lags with each extension, as in the illustrative extend/lag procedure 43, can incorporate more time information while minimally increasing the number of channels/features. This in turn can augment the covariance matrix in the case of using a joint diagonalization technique, such as AJDC filters, to separate sources. In contrast, using single lags does not allow for features to be computed over time.

In a nonlimiting example of the extend/lag procedure 43, The input EMG data X is first divided into non-overlapping windows of size L. This binning step ensures that the data is segmented into manageable chunks for subsequent processing. The binned data is represented as X_{binned}∈ [symbol] ^{W×C×L}, where C is the number of channels and W is the number of windows. To incorporate temporal information, the data is extended using lagged versions of each channel. This augmentation creates an extended EMG dataset \tilde{X}_{binned}∈ [symbol] ^{W×(C·R)×L}, where R is the extension factor. The extend-lag procedure increases the ratio of observations to sources, improving the conditioning of the source separation problem. By embedding the EMG data 40 into a higher-dimensional space, this approach captures both spatial and temporal dependencies, which are advantageous for resolving sources with overlapping activity.

In an operation 44, covariance matrices are computed from the input training EMG data 40 to generate a smooth differentiable manifold on which the original data lies (n_domains×n_samples×n_features×n_features). In one suitable approach, the operation 44 calculates Fourier cospectral covariance matrices to enhance separation of sources in the spectral domain. Alternative methods for the operation 44 include the empirical covariance with/without regularization, or feature-wise kernel methods (such as the Laplacian, sigmoidal, or cosine kernels, or so forth). In the illustrative embodiment of FIG. 2 which employs the optional extend/lag procedure 43, the augmented covariance matrix advantageously combines spatial covariance with temporal information, effectively embedding the original EMG dataset 40 into a higher-dimensional space. This embedding enhances the separability of sources by capturing their temporal dynamics and spatial structure. The process is mathematically equivalent to constructing a delay-embedded dataset as follows:

$$\tilde{X} ( t ) = {[ x ( t ) , x ( t - τ ) , … , x ( t - ( R - 1 ) τ ) ]}^{T} \qquad ( 1 )$$

where τ is the lag parameter, R is the embedding dimension, and T is the transpose operator. By incorporating temporal information, the extend-lag procedure improves the robustness of the decomposition algorithm, particularly in scenarios where sources have overlapping spatial patterns or similar spectral characteristics. This augmentation is advantageous for capturing non-stationary and dynamic characteristics of the EMG signals 40, thereby facilitating identification of motor unit activity.

In an optional operation 46, the covariance matrices are normalized and averaged across domains (in implementations in which the training EMG dataset 40 is acquired across multiple subjects and/or sessions). The subsequent covariance matrices are whitened to center the data (with or without a dimension reduction). In an operation 48, AJDC filters are computed. In one suitable approach, diagonal filters are approximated using an approximate joint diagonalization algorithm. In one suitable computation approach, Quasi-Newton joint approximation is used to increase speed and separability of sources. BSS filters 50 which in this embodiment include both forward filters 50-1 and backward filters 50-2 are computed in the operation 48 from the approximated diagonal filters and whitening filters to transform to the source space (using the forward filters 50-1) and from source space back to the time domain (using the backward filters 50-2).

Additionally, the BSS filter fitting 42 determines (i.e., fits) one or more thresholds 52 for detecting artifacts in the EMG data. The further processing 32 (see FIG. 1) can automatically detect outlier sources (which are expected to be artifacts) based on the preset thresholds 52 of what constitutes an outlier source. Kurtosis, time-correlation, median absolute deviation (MAD), sparsity, and source correlation are some suitable methods for automatically distinguishing artifact sources from true signals. In some examples, a time-correlation threshold can be fitted to capture sources with repeating artifacts (e.g. in the case of a repeated artifact from NMES pulses produced by the NMES stimulator 24 if the training EMG data 40 includes training EMG data collected during interleaved EMG measurement/NMES stimulation intervals). A source correlation threshold can be fitted to detect sources that are highly correlated, which can help find artifacts from electrical stimulation contained in multiple sources. A sparsity threshold can be fitted to detect localized artifact sources. The fitting of these threshold can be done using the training EMG data 40 with the subject artifact sources labeled, and the thresholds are optimized to optimally distinguish the labeled artifacts from the remainder of the training EMG data.

It is again noted that the BSS filter fitting method described above with reference to illustrative operations 44, 46, and 48 is for a nonlimiting illustrative example in which the BSS 30 of FIG. 1 employs AJDC matrixes as the BSS method. More generally, the BSS filter fitting 42 uses the training EMG data 40 to fit BSS filters 50 suitable for the type of BSS method to be used in the operation 30 to identify motor unit sources. As some further examples, the BSS filter fitting 42 can fit BSS filters for an AJDC method (as in the example illustrated), an ICA method, a principal component analysis (PCA) method, a non-negative matrix factorization method, a low-complexity coding and decoding method, or other chosen BSS method. The BSS filter fitting operation 42 obtains suitable BSS filters 50 for the chosen BSS method of performing the operation 30 of FIG. 1 for transforming the EMG data to separated sources, and also fits thresholds 52 for use in the further processing 32 of FIG. 1.

BSS can advantageously elucidate where the artifact source is localized in reference to the original EMG data. In the case of FES, the artifact originates from the electrodes that produce electrical stimulation. In comparison to the rest of an HD-array of electrodes, this is localized in a small sparse region of the array. Therefore, a sparsity threshold can be used to determine which sources extracted by the BSS 30 are sparse, indicating a likely source of artifact, which can be suppressed. Since the sources extracted by the BSS 30 originate at the stimulation patterns, it is likely that this entire source selectively encodes stimulation artifact and can thus be removed. This should not remove any of the true EMG signal from the associated EMG channels. It merely suppresses the artifact from the original EMG data by suppressing the artifact source associated that is only active during artifact periods.

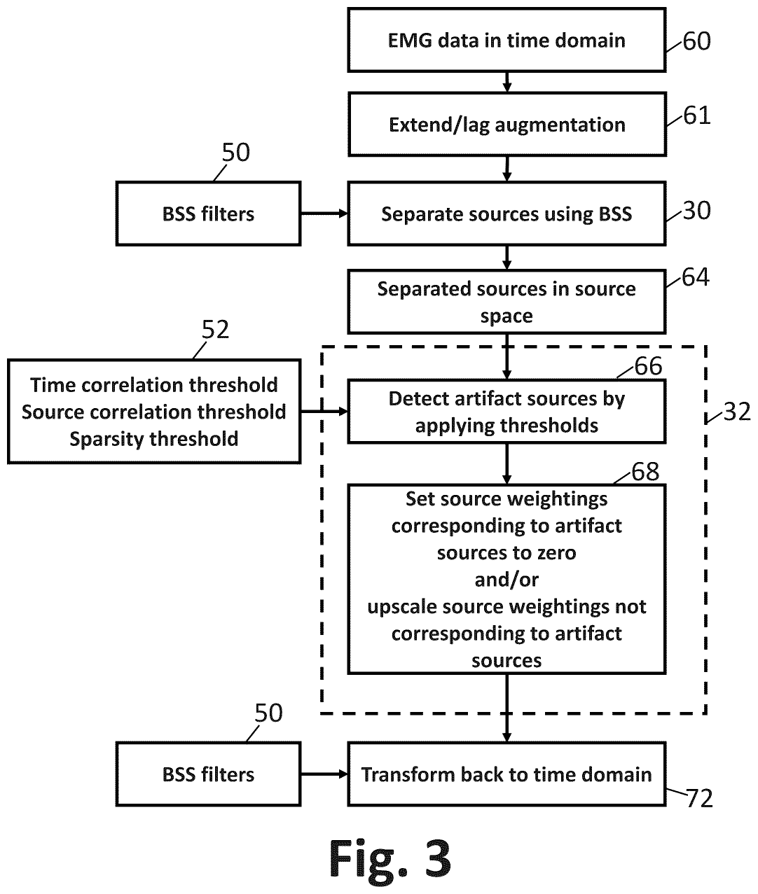

With reference now to FIG. 3, an illustrative example of an embodiment of the operation 30 of separating sources from the EMG data (see FIG. 1) is shown. EMG data 60 in the time domain is transformed in the operation 30, which uses the fitted BSS filters 50 to perform the transformation, thereby producing EMG data represented as separated sources in source space 64. As previously noted, the BSS operation 30 may employ any suitable BSS method, such as an AJDC method, an ICA method, a PCA method, a non-negative matrix factorization method, a low-complexity coding and decoding method, or other chosen BSS method. If the BSS filter fitting of FIG. 2 employed the optional extend/lag procedure 43, then a corresponding extend/lag procedure 61 is applied to the EMG data 60 before performing the source separation 30. The separated sources 64 are expected to include motor units (i.e., MUAP action potentials), but may also include artifact sources. Further processing 32 is then performed to extract the MUAP's from the separated sources. In an illustrative example of the further processing 32, in an operation 66, artifact sources are detected using the fitted thresholds 52. The time-correlation threshold is applied to identify sources with repeating artifacts, such as from repetitive NMES pulses. The source correlation threshold is applied to identify sources that are highly correlated, which can help find artifacts from electrical stimulation contained in multiple sources. The sparsity threshold is applied to detect localized artifact sources.

To suppress or remove the artifact sources detected by the operation 66, an operation 68 sets source weightings in source space 64 corresponding to artifact sources to zero, and/or upscales source weightings of the EMG data in source space 64 not corresponding to artifact sources. The approach of upscaling source weightings not corresponding to artifact sources amplifies good sources (i.e., sources without artifacts exceeding one or more of the thresholds 52) by increasing the weighting of the sources when transforming back into the original data space. An example implementation entails calculating the fisher score or mutual information to determine discriminability of sources in reference to training labels. The ranking of sources by information can be used to weight the sources along the diagonals of the backward filters. It is also contemplated to directly calculate features and decode from source space if desired. The weightings produced by the operation 68 are used in combination with the backward filters to transform the data back into time domain in operation 72, yielding the EMG signal reconstructed with artifacts suppressed due to the source weightings set in operation 68.

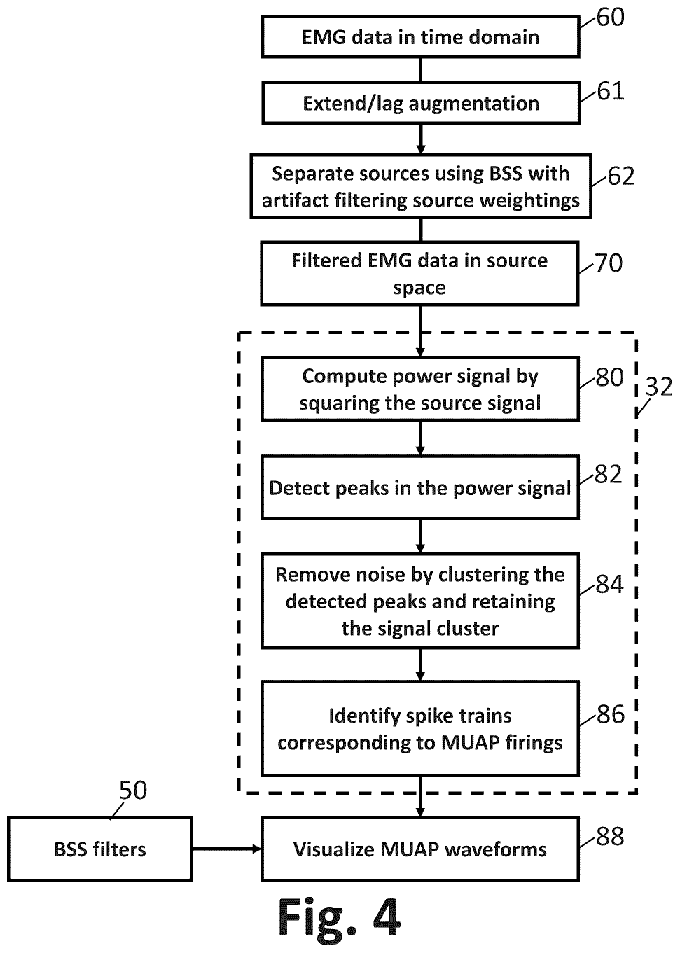

With reference now to FIG. 4, an illustrative example of an embodiment of the MUAP decomposition process 32 of FIG. 1 is shown. The illustrative MUAP decomposition process 32 of FIG. 4 operates in source space; accordingly, it receives the EMG data in time domain 60 and transforms the EMG data into source space in an operation 62, optionally with source weighting adjusted by operation 68 of FIG. 3, to produce filtered EMG data in source space 70. If the BSS filter fitting of FIG. 2 employed the optional extend/lag procedure 43, then the corresponding extend/lag procedure 61 is applied to the EMG data 60 before performing the source separation 62. As discussed with reference to FIG. 3, the filtered EMG data in source space 70 advantageously has artifacts suppressed or removed. To perform the MUAP decomposition 32, in an operation 80 the signal power is computed by squaring the source signal. In an operation 82, peaks are detected in the power signal based on optional height and inter-spike interval requirements. It is noted the operations 80 and 82 are performed for each source signal in the filtered EMG data in source space 70; that is, power signals corresponding to source signals in the filtered EMG data in source space 70 are computed by squaring the respective source signals. In an operation 84, peak signals are clustered using k-means (or another suitable clustering algorithm) into two groups to distinguish signal from noise, and peak indices within the signal cluster are kept and the detected noise peak indices are discarded.

In an operation 86, spike trains corresponding to the MUAPs are identified. In one approach, binary spike trains or sample arrays are identified based on peak index, and are then returned in a suitable format such as binned (e.g. one spike per MU per bin) or within bins in spikes per bin samples. From there, the cumulative spike train (CST) can be determined across all motor unit sources or for motor units that fire selectively during different movements. The smoothing of the CST is referred to as neural drive, which can be used to train decoders. Alternatively, other embodiments find the power spectrum of motor unit firing (e.g. in the beta band) to indirectly obtain motor cortex commands from EMG data. Optionally, average coherence of randomly sampled CSTs can additionally provide information about the input command from motor cortex as well.

The output of the operation 86 is the decomposed MUAPs represented as spike trains in source space. The spike trains determine when motor unit sources fire in time. Typically, the spikes are treated as binary (on/off), although more complex interpretations are also contemplated. The decomposed MUAPs can be used for various purposes, such as the intent decoding 34 and/or neuromuscular assessment 36 of FIG. 1. Additionally or alternatively, the decomposed MUAPs represented as spike trains in source space can be visualized 88. Based on the firing timings, waveforms can be extracted from the original EMG signal. In another approach, the motor unit sources can be visualized using a heatmap of the BSS filter 50.

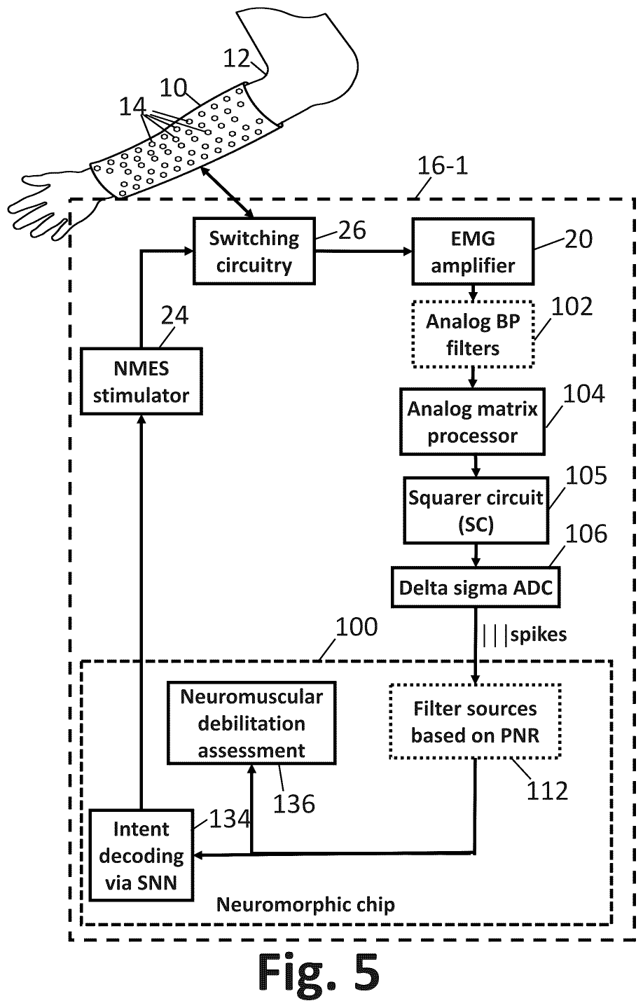

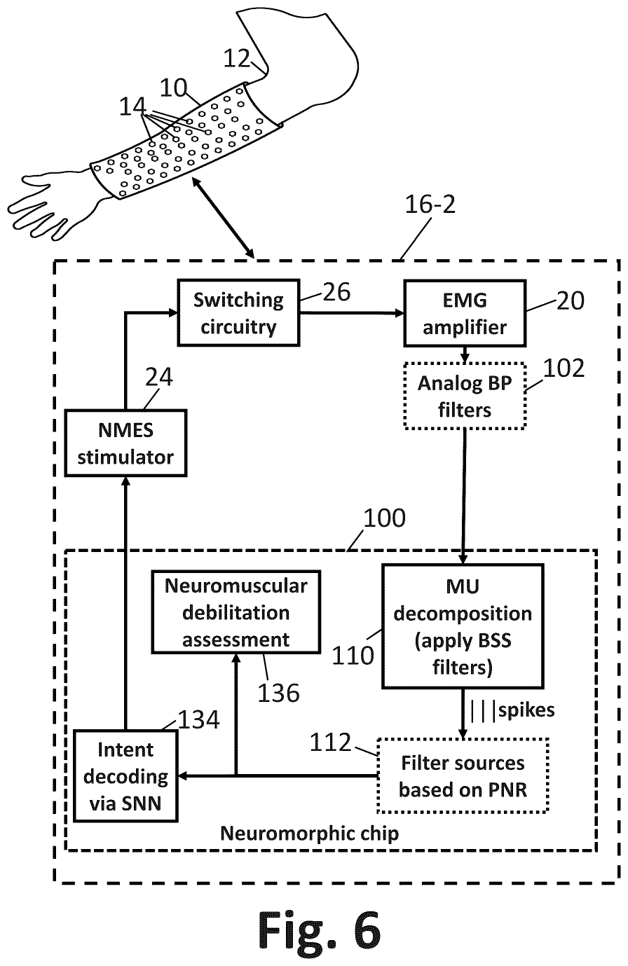

With reference now to FIGS. 5 and 6, an EMG measurement system with optional NMES capability according to variant embodiments are diagrammatically illustrated. The systems of FIGS. 5 and 6 are similar to that of FIG. 1, and each includes the garment 10 with electrodes 14 worn on the anatomical region 12, and variant electronics 16-1 (FIG. 5) or electronics 16-2 (FIG. 6) that also include the EMG amplifier 20, optional NMES stimulator 24, and switching circuit 26 (which can be omitted if the NMES stimulator 24 is omitted).

However, the variant electronics 16-1 and 16-2 of the embodiments of FIGS. 5 and 6 differ from the electronics 16 of the embodiment of FIG. 1 in that the ADCs 22 of the embodiment of FIG. 1 are omitted, and the EMG processing is implemented using a neuromorphic chip 100 which operates in the analog domain on the analog EMG signals after optional bandpass filtering by optional analog bandpass filters 102 to filter out unwanted frequency components.

In the embodiment of FIG. 5, conversion of the analog EMG signals to spike signals is performed using an analog matrix processor (AMP) 104, an analog squarer circuit 105 (implementing the squaring operation 80 of FIG. 4), and ADCs 106 which may be implemented as event driven ADC, e.g. as delta-modulator ADCs 106.

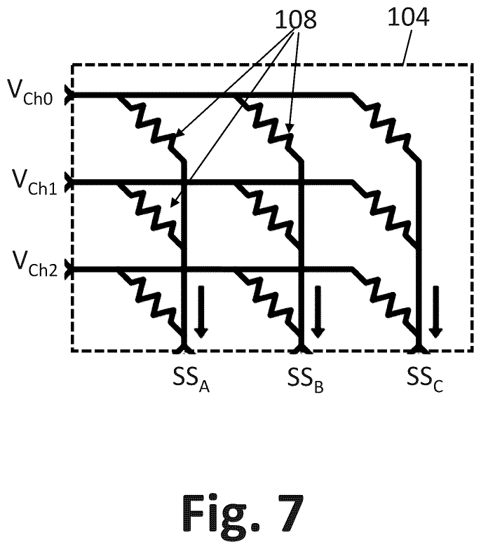

With continuing reference to FIG. 5 and with further reference to FIG. 7 which shows the analog matrix processor 104 in further detail, the human nervous system processes information via synapses between neurons triggered by action potentials. In the same way, the system of FIG. 5 transfers information via MUAPs in an event-based manner. To do this, the EMG signals are first transformed into an event-based signal. To do this, the analog EMG signals are first transformed into an event-based signal. This is done in the analog domain by the optional bandpass filters 102 and the analog matrix processor 104 to substantially reduce power consumption. The analog matrix processor 104 takes the input EMG signal (after amplification by the EMG amplifiers 20 and optional bandpass filtering by the analog bandpass filters 102) and transforms it into a source space of MU activity. The analog matrix processor 104 takes the EMG signal voltages (e.g., indicated analog EMG voltage signals V_{Ch0}, V_{Ch1}, V_{Ch2}, . . . ) as an input, and performs matrix multiplication in the analog domain. The weights for the matrix multiplication are stored as resistances 108, with the resultant output as spike signals (e.g., indicated spike signals SS_{A}, SS_{B}, SS_{C}, . . . ) in the analog domain, e.g., represented as analog electric currents. The forward filters suitably comprise whitening and blind source separation decomposition method, and can be fit offline using any suitable MUAP decomposition 32, such as the BSS filter fitting approach described with reference to FIG. 2. Passing through the squarer circuit 105 provides the power of source activity. The ADCs 106 then convert the analog MUAP signals to digital MUAPs. The ADCs 106 may be implemented as event driven ADC, e.g. as delta-modulator ADCs. In the embodiment of FIG. 5, the output of the ADCs 106 serve as input to a neuromorphic chip 100 that performs further processing. Spatial information of source location based on BSS filters 50 can be used to train a spiking neural network (SNN) implemented in the neuromorphic chip 100 using convolutional layers. Thus, in the embodiment of FIG. 5 the MUAP decomposition is performed in analog by the analog matrix processor 104 and squarer circuit 105, which then feeds into the neuromorphic chip 100 already quantized (as spikes). Note that during training, sources can also be removed based on whether or not they are artifacts, e.g., using fitted thresholds 52 as previously described. FIG. 5 illustrates processing with artifacts already removed.

With reference to FIG. 6, in this embodiment the electronics 16-2 again include the EMG amplifier 20 feeding optional analog EMG data into the analog bandpass filters 102 to filter out unwanted frequency components. The subsequent analog components 104, 105, and 106 of the electronics 16-1 of the embodiment of FIG. 5 are omitted in the electronics 16-2 of the embodiment of FIG. 6, and instead the neuromorphic chip 100 in an operation 110 directly encodes spikes based on input data as a first step. A similar process is used, in which the BSS filters 50 are stored and applied in inference, but here using the neuromorphic chip 100. During training, sources can also be removed based on whether or not they are artifacts, e.g., using fitted thresholds 52 as previously described. FIG. 6 illustrates processing with artifacts already removed.

In the embodiments of both FIG. 5 and FIG. 6, optional filtering based on pulse-to-noise ratio (PNR) in an operation 112 implemented by the neuromorphic chip 100. Following this, intention is decoded in an operation 134 via a spiking neural network (SNN) encoder implemented on the neuromorphic chip 100 (for example, an SNN autoencoder), and/or in an operation 136 neuromuscular debilitation assessment via MUAP features and/or an SNN autoencoder is performed in an operation 136 implemented on the neuromorphic chip 100. The operations 134 and 136 correspond to respective operations 34 and 36 of previous embodiments, but implemented on the neuromorphic chip 100 as disclosed herein.

In the embodiment of FIGS. 5 and 6, the measured analog EMG signal is the direct neural command to muscles via motor unit action potentials. Neuromorphic hardware, e.g. the neuromorphic chip 100, can be used to train models to classify the information. For example, a Loihi neuromorphic chip (available from Intel Corporation) can be used as the neuromorphic chip 100 to process the input spikes via a spiking neural network (SNN). Depending on the application, whether decoding via the decoder 134 to actuate an effector (e.g. robotic arm or functional electrical stimulation) or performing neuromuscular debilitation assessment 136 such as biomarker analysis (e.g. fatigue, co-contraction, spasticity, neural drive), the SNN implemented by the neuromorphic chip 100 can be pre-trained and fixed or adapting to produce the desired output.

Using neuromorphic hardware (i.e., the neuromorphic chip 100) substantially reduces energy expenditure, increases inference speed, provides robustness against artifacts/failures when compared with implementation of the processing in the digital domain as in the embodiment of FIG. 1. Using neuromorphic hardware also allows for on-the-fly learning via neuron firing rules. The reduced electrical power consumed by the neuromorphic chip 100 facilitates construction of a completely mobile system for usage domains such as military in-the-field operations, use by athletes, use in space, and so forth.

One approach for analog processing of analog EMG signals is to convert as-measured EMG to spikes based on applying a suitable threshold, and then feeding the thresholded EMG data into a neuromorphic chip. This, however, does not have a physiological foundation and only considers global EMG activity. Therefore, as movements become more complex, the number of classes increases, or the application changes (e.g. performing a biomarker assessment), this EMG thresholding approach has difficulty handling the additional complexity.

By contrast, the embodiment of FIG. 5 transforms the as-measured EMG signal output by the EMG amplifiers 20 (and optionally filtered by the analog bandpass filters 102) to motor unit source domain using BSS filters 50 via the analog matrix processor 104 and squaring the signal before detecting spikes to feed into the neuromorphic chip 100. In FIG. 6, the MU decomposition 110 is implemented on the neuromorphic chip 100. In these embodiments, a better representation can be used for training the SNN implemented by the neuromorphic chip 100. The analog matrix processor 104 or implementation of the operation 110 on the neuromorphic chip 100 advantageously has low power consumption, and can directly implement deep learning models that are trained using traditional computing methods.

Combining analog processing with neuromorphic computing performed by the neuromorphic chip 100 advantageously consumes low levels of energy. This benefit becomes increasingly more pronounced as the number/density of electrodes 14 increases, as for example if the garment 10 is a whole-body garment with full-body electrode arrays. Performing the MUAP decomposition in the analog domain using a CPU with collection of large data arrays becomes more challenging as the amount of data being processed increases, leading to latency issues, and higher power consumption. These issues are particularly problematic in mobile applications in which the system of FIG. 5 is a wearable system with the electronics 16-1 implemented as (for example) a waistbelt-worn electronics unit. In the embodiments of FIGS. 5 and 6, these problems are overcome because only events which by nature are sparse are captured, enabling low power operation.

In the example of FIGS. 5 and 6, processing in the analog domain using the neuromorphic chip 100 is applied for processing EMG signals, which are expected to biologically encode spike signals as a consequence of neural firing of biological neurons which produces the EMG. More generally, other measured biological signals such as electroencephalography (EEG), heart rate, galvanic skin response (GSR), photoplethysmography (PPG), neuron local field potentials/spiking activity measured via invasive microelectrode arrays, and so forth are expected to have similar spiking, and so the approach of FIG. 5 or of FIG. 6 can be analogously used on such signals to convert them to source domain, from which a source power is computed, and subsequent spikes are determined in the analog domain. The neuromorphic chip 100 classifies or tracks biologically relevant biomarkers in a low-powered system. Hence, the disclosed implementations using the neuromorphic chip 100 are suitable for low-power and low-latency mobile biological monitoring of brain waves via EEG, heart rate, GSR, PPG, and/or so forth.

With reference back to FIGS. 1, 5, and 6, the intent decoding 34 or 134 operates on the MUAPs produced by the MUAP decomposition 32 implemented in the digital domain (as in the system of FIG. 1), or in the analog domain (as in the systems of FIGS. 5 and 6). Various approaches can be used for this decoding. A challenge in implementing the intent decoding 34 or 134 is that the intent is more directly related to the motor cortical signals, with the EMG signals being produced through transmission and processing of the motor cortical signals.

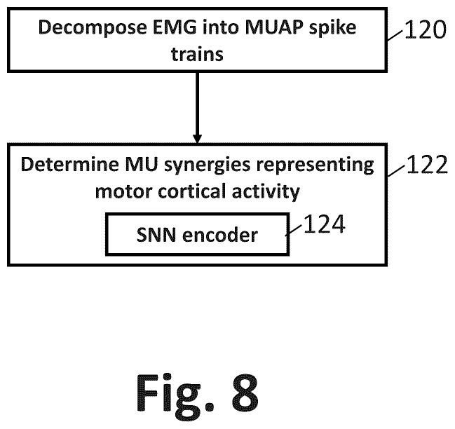

With reference to FIG. 8, in the following, approaches are described for inferring motor cortical activity based on the resulting MU spiking input to muscles. In an operation 120, EMG data is decomposed into MU spike trains via an MU decomposition method such as the processing 62 and 32 of FIG. 4 (with optional extend/lag augmentation 61) or corresponding processing of the embodiments of FIG. 5 or FIG. 6 as previously described. Next, in an operation 122 a spiking neural network (SNN) encoder 124 uncovers latent representations of the MU spiking signal. As an example, an autoencoder structure may be used to minimize the error between the input and output of the network to uncover plausible up-stream neurons in cortex. Optionally, contrastive learning can be employed to further uncover encoded latent spiking activity associated temporally and/or with behavior. The SNN encoder 124 may include both excitatory and inhibitory connections. These latent representations or modules of common MUs are sometimes referred to as motor unit (MU) synergies. The central nervous system (CNS) activates MU modules to produce movement. Some approaches for deriving MU synergies utilize dimensionality reduction techniques on smoothed cumulative MU spike trains to describe MU modules. The approach of FIG. 8 processes only the spiking data to infer the common synaptic input spike trains. Neurons can be approximated with internal dynamics (e.g. a leaky integrate and fire model, etc.). This allows the SNN encoder 124 to learn plausible synaptic weights to an unknown number of preceding descending neurons based on neuron firing dynamics. Additionally, there is the potential to extrapolate additional inputs (rather than reduce dimensions with the encoder). While this violates the hypothesis of the CNS activating MU synergies, as increased decoding performance is obtained when assuming additional inputs from the motor cortex suggesting there may be relevant cortical activity missed when assuming the CNS reduces the dimensionality of control.

The operation 122 and the SNN encoder 124 may be implemented on the digital electronic processor 28 in the embodiment of FIG. 1, or alternatively may be implemented on the neuromorphic chip 100 of the embodiment of FIG. 5 or FIG. 6. Implementation on the neuromorphic chip 100 advantageously provides low power consumption and low latency, again facilitating implementation in a mobile system with the electronics 16-1 or 16-2 implemented as (for example) a waistbelt-worn electronics unit.

The approach of FIG. 8 infers the cortical input to the descending motor unit firing by enforcing neuronal network dynamics inspired by physiology. A SNN is capable of uncovering synaptic weights between neurons with internal state dynamics. By using the SNN encoder 124, an intermediate latent space can uncover the common synaptic input with known spike timings. As a result, this provides a more flexible control signal for a human-machine interface (HMI) and (when used for debilitation assessment 36) a more holistic biomarker of disease state. Advantageously, the approach of FIG. 8 enables “backward” inference of the cortical neural signals that produced the measured EMG signals, by inferring the common synaptic input to the extracted MUs. This input to MUs serves as a proxy for cortical activity. Advantages of estimating cortical activity from a peripheral interface include allowing for a higher degree of freedom when used for control, in addition to a potentially more informative biomarker of disease state when used for debilitation assessment 36.

The method of FIG. 8 for inferring cortical activity from measured peripheral EMG data (e.g., as measured from a forearm 12 by the sleeve 10) advantageously retains time-frequency characteristics of the predicted input to the MU neurons. The method takes advantage of spiking neural networks (SNNs) to uncover cortical activity that activates the MU firings decomposed from the EMG recording. Inspired by biology, SNNs incorporate neuron dynamics similar to what have been observed in biological neuron recordings. For example, a simple leaky integrate-and-fire model can be used for each neuron in the SNN. Input spikes from the axon of the preceding neuron feed into the dendrites of a MU neuron. The frequency of spikes increases the internal membrane voltage of the MU neuron until it reaches a threshold and a spike is generated. The method of FIG. 8 infers the input into the MU neurons that result in the MUAP spikes obtained by the processing 32. Alternative more complex neuron dynamic models can be used as well in the network with both excitatory and inhibitory connections (e.g. resonate-and-fire, Hodgkin and Huxley, Current Based Leaky Integrate and Fire (CUBA), etc.).

The output of a SNN consists of neuronal spike trains. Therefore, to uncover the input to MU neuron firings, the SNN encoder 124 can be used to either reduce or expand the MU firing activity. The MU spike trains are fed into the SNN encoder 124 with a hidden layer of N dimensions and subsequently an output layer with the same size output as the MU spike train. The hidden layer, thus, includes the input to the MU spike trains and is an estimation of the cortical neuron activity resulting in the measured MU spike trains. As in the case with an artificial neural network (ANN) encoder, the error between the input and output of the SNN encoder 124 should be minimized to learn the weights. The mean squared error between spike trains can be used. Alternatively, a spike time-based loss can be used for the error function. One suitable approach utilizes the spike time-based loss from the open-source LAVA toolbox. Optionally loss functions may employ contrastive learning to further find similar latent components associated with temporal or behavioral characteristics of the MU spike trains. The SNN is trained using Spike Layer Error Reassignment in Time (SLAYER) to account for the non-differentiable spike generation inherent in SNNs. Synaptic weights and delays can then be learned directly by minimizing the error function. Spike timing and synaptic connections are learned in order to retain interpretability of the cortical features/firing extracted (e.g. spatial weighting of synergistic MUs). Once the synaptic connections are learned, the SNN encoder can be used in inference to infer cortical activity in real-time. These synaptic weights can be continuously updated with neuron firing rules for continual learning if desired.

Overall, initial results of actually-performed motor cortical signal inference in accordance with the method of FIG. 8 showed that the SNN encoder 124 is generalizable, as the input/output spike trains were 98.2% similar in the hold-out test set.

In further embodiments, a motor cortical activity estimation method includes measuring EMG data emanating from the anatomical region 12, decomposing 32 the EMG data into MUAPs, and determining MU synergies representing motor cortical activity from the MUAPs using the SNN encoder 124. In some such embodiments, the MU synergies are determined using the neuromorphic chip 100 that implements the SNN encoder 124. In some such embodiments, the decomposing 32 includes performing analog matrix multiplication using the analog matrix processor 104 to transform the EMG data into analog spike signals in a source space, and decomposing the analog spike signals into the MUAPs using the neuromorphic chip 100.

One application of inferring cortical activity is to use this information for control of NMES applied by the NMES stimulator 24. Here, the intent decoding 34 is based on the inferred motor cortical signals. In the actually-performed motor cortical signal inference, the prediction probabilities of the classification problem over time were also tested. A SNN classifier was trained to classify 4 hand/wrist movement classes (lateral pinch supination, medium wrap, parallel extension, and quadpod) along with a rest class in 100 ms bins. When reducing the spike trains to motor unit synergies, there was a reduction in bin-wise accuracy. However, when increasing dimensions to infer a larger input to MU activity, decoding accuracy increased compared to decoding directly from the MUs. This is not necessarily the case for all datasets, and may be a result of the SNN classifier, but in the performed tests the confidence of the decoder increased with an increasing number of input neurons.

Another application of inferring the cortical activity is the neuromuscular debilitation assessment 36, for example identifying a potential biomarker of disease state (e.g. monitoring motor function of stroke survivors or a diagnostic of ALS). Using typical methods from MU analysis, the neuron firing coherence in different frequency bands can be uncovered. These features can then be used to make predictions and stratify subjects based on function.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. An electromyography (EMG) measurement device comprising:
a garment configured to be worn on an anatomical region of an associated wearer;
a plurality of electrodes arranged on the garment to contact skin of the anatomical region when the garment is worn on the anatomical region of the associated wearer; and
electronics configured to perform motor unit action potential (MUAP) decomposition on the EMG data, the electronics including:
an EMG amplifier operatively connected with the electrodes to measure analog EMG data emanating from the anatomical region, and
circuitry configured to decompose the analog EMG data into MUAPs.

2. The EMG measurement device of claim 1, wherein the circuitry includes:
an analog matrix processor configured to perform analog matrix multiplication to transform the analog EMG data into source signals.

3. The EMG measurement device of claim 2, wherein the circuitry further includes:
an analog squarer circuit configured to compute power signals by squaring the source signals output by the analog matrix processor; and
delta sigma analog-to-digital converters configured to convert the power signals to analog spike signals.

4. The EMG measurement device of claim 3, wherein the circuitry further comprises:
a neuromorphic chip configured to process the analog spike signals to determine volitional intent using a spiking neural network (SNN) encoder.

5. The EMG measurement device of claim 1, wherein the circuitry further comprises:
a neuromorphic chip configured to perform a neuromuscular debilitation assessment based on the analog spike signals.

6. The EMG measurement device of claim 1, wherein the circuitry includes:
a neuromorphic chip configured to transform the analog EMG data into analog spike signals using blind source separation.

7. The EMG measurement device of claim 6, wherein the neuromorphic chip is further configured to process the analog spike signals to determine volitional intent using a spiking neural network (SNN) encoder.

8. The EMG measurement device of claim 1, wherein the circuitry f is configured to decompose the analog EMG data into MUAPs by operations including:
performing an extend/lag procedure to with a lag greater than 1 to generate an extended EMG dataset; and
decomposing the extended EMG dataset into MUAPs using blind source separation.

9. A motor cortical activity estimation method comprising:
measuring electromyography (EMG) data emanating from an anatomical region;
decomposing the EMG data into motor unit action potentials (MUAPs); and
determining motor unit (MU) synergies representing motor cortical activity from the MUAPs using a spiking neural network (SNN) encoder.

10. The method of claim 9, wherein the MU synergies are determined using a neuromorphic chip that implements the SNN encoder.

11. The method of claim 10, wherein the decomposing includes:
performing analog matrix multiplication using an analog matrix processor and digitization using analog to digital converters to transform the EMG data into analog spike signals in a source space.

12. The method of claim 9, further comprising at least one of:
operating a neuromuscular electrical stimulation (NMES) stimulator based on the MU synergies to deliver functional electrical stimulation to cause movement of the anatomical region; and/or
performing a neuromuscular debilitation assessment based on the MU synergies.

13. The method of claim 9, wherein the decomposing of the EMG data into MUAPs includes:
performing an extend/lag procedure to with a lag greater than 1 to generate an extended EMG dataset; and
decomposing the extended EMG dataset into MUAPs using blind source separation.

14. An electromyography (EMG) data processing device, the EMG data processing device comprising:
analog source separation circuitry configured to transform analog EMG data into source signals using blind source separation; and
analog spike signal generation circuitry configured to convert the source signals into analog spike signals;
wherein at least some of the analog spike signals correspond to motor unit action potentials (MUAPs).

15. The EMG data processing device of claim 14 wherein the analog source separation circuitry comprises:
an analog matrix processor configured to perform analog matrix multiplication to transform the analog EMG data into source signals; and
an analog squarer circuit configured to compute power signals by squaring the source signals output by the analog matrix processor.

16. The EMG data processing device of claim 15 wherein the analog spike signal generation circuitry comprises:
delta sigma analog-to-digital converters configured to convert the power signals to analog spike signals.

17. The EMG data processing device of claim 14, wherein the analog source separation circuitry and the analog spike signal generation circuitry comprise
a neuromorphic chip configured to transform the analog EMG data into analog spike signals corresponding to source signals using blind source separation.

18. The EMG data processing device of claim 14, further comprising:
a neuromorphic chip configured to process the analog spike signals to determine volitional intent using a spiking neural network (SNN), and/or to perform a neuromuscular debilitation assessment based on the analog spike signals.

19. The EMG data processing device of claim 14, further comprising:
analog filtering circuitry configured to select the analog spike signals corresponding to MUAPs from the analog spike signals output by the analog spike signal generation circuitry.

20. The EMG data processing device of claim 19, wherein the analog filtering circuitry selects the analog spike signals corresponding to MUAPs based on pulse-to-noise ratios (PNRs) of the analog spike signals output by the analog spike signal generation circuitry.
